(19) United States
(12) Plant Patent Application Publication
(10) Pub. No.: US 2003/0056264 P1
(43) Pub. Date: Mar. 20, 2003

(54) Chrysanthemum plant named 'Baja'

(76) Inventor: Mark Roland Boeder, J H Den Haag (NL)

Correspondence Address: Mark K. Dickson, HOWREY SIMON ARNOLD & WHITE, LLP, 750 Bering Drive, Houston, TX, 77057-2198, US

(21) Appl. No.: 09/957,495

(22) Filed: Sep. 19, 2001

(51) Int. Cl. A01H 5/00

(52) U.S. Cl. PLT/289

(57) ABSTRACT

A new variety of Chrysanthemum plant named ‘Baja,’ having a unique ability to produce one very large flower on a disbudded single stem, a formate known as Sombrero. The flowers are spider type and are white in colour with a yellow centre.

### BACKGROUND OF THE INVENTION

The present invention comprises a new and distinct cultivar of Chrysanthemum botanically known as Dendranthema grandiflora, and referred to by the cultivar name ‘Baja’.

‘Baja’, identified as 20300-0 originated from a cross made by Mark Boeder in a controlled breeding program in Chichester, West Sussex, United Kingdom. The female parent of the new variety is ‘Fontana,’ U.S. Plant Pat. No. 6948, and the male parent is the variety known as ‘Joy Time.’ The new variety ‘Baja’ has been asexually reproduced by vegetative cuttings in Chichester, West Sussex, United Kingdom and the distinguishing characteristics are retained through successive generations of asexual reproduction.

### BRIEF SUMMARY OF THE INVENTION

‘Baja’ is a pot type of Chrysanthemum plant variety having white decorative spider type flowers with yellow centre.

### Comparison with Parent

Plants of the new Chrysanthemum variety ‘Baja’ are similar to the parent cultivar ‘Fontana’ in plant habitat and growth rate. In side-by-side comparisons in Chichester, West Sussex, United Kingdom, under commercial practice, plants of the new Chrysanthemum variety ‘Baja’ compared to plants of the parent cultivar ‘Fontana’ in the following characteristics.

1. The new variety ‘Baja’ produces white decorative spider type flowers, whereas the parent cultivar ‘Fontana’ produces yellow spider type flowers.

2. Plants of the new variety ‘Baja’ have larger and taller inflorescence than plants of the parent cultivar ‘Fontana.’

3. ‘Baja’ has more Ray florets and long corolla tubes, compared with the parent cultivar ‘Fontana.’

### Comparison with Other Varieties

Plants of the new Chrysanthemum variety ‘Baja’ are dissimilar to the cultivar ‘Kodiac’ in plant habitat and growth rate. However, in side-by-side comparisons in Chichester, West Sussex, United Kingdom, under commercial practice, plants of the new Chrysanthemum variety ‘Baja’ differed from plants of the cultivar ‘Kodiac’ in the following characteristics.

1. The new variety ‘Baja’ produces white decorative spider type flowers, whereas the cultivar ‘Kodiac’ produces white quill flowers.

2. Plants of the new variety ‘Baja’ have larger and taller inflorescence than plants of the cultivar ‘Kodiac.’

3. The new variety ‘Baja’ has more incurving Ray florets than ‘Kodiac,’ and a response time of five days slower than ‘Kodiac.’

### BRIEF DESCRIPTION OF ILLUSTRATIONS

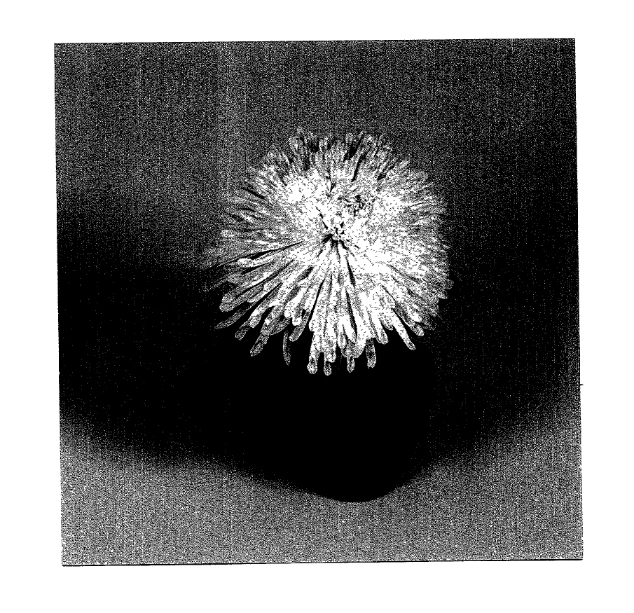

Typical specimens of the plant and flowers for the new Chrysanthemum variety ‘Baja’ are shown in the accompanying photograph. The colours shown are as true as possible within the usual limits of this kind of illustration. FIG. 1 is a whole plant view of the new variety ‘Baja’ grown in a pot. The plant shown in the illustration is 58 days from start of Short Days.

### DETAILED BOTANICAL DESCRIPTION

The following description of the new Chrysanthemum variety ‘Baja’ is of plants grown in a greenhouse in Chichester, West Sussex, United Kingdom in the month of August. The cultivar has not been observed under all possible environmental conditions. The phenotype may vary significantly with variations in the environment such as temperature, length of day and light intensity, without any variance in genotype. The commercial classification of the new variety is a pot Chrysanthemum.

Plants of the new variety have been grown successfully under temperature conditions averaging about 16° C. at night and about 24° C. to 25° C. during the day under light conditions of about 5,000 to 6,000 foot candles. The plants respond well to the use of growth retardant, such as three B9 treatments at about 2000 ppm. To produce a commercial product the plants may be disbudded with the centre bud left in place. The typical container size for commercial growth is 9 cm. It has been observed that the shelf life of the new variety is about 28 days with a response time of about 8.5 weeks. The new variety is suitable for growth in a temperature range of 16° C. to 25° C.

The new variety may be produced as a Sombrero disbud. The following description is with respect to a plant produced as a disbud. In the description of this new Chrysanthemum variety, colour values have been taken from The Royal Horticultural Society Colour Chart (R.H.S.C.C.).

### Plant

Plant type: Pot

Height: Short 12 to 15 cm

Width: Near 20 cm

Branching characteristics: Angular

Stem Colour: 146 A

Response Time: 58 days

Vigour: Medium height

Shelf Life: Near 28 days

Disease: No observations

Pest: No Observations

Growth retardant type and treatment: B9 85% 3 applications of 2 gram/litre at 2 days, 21 days and 30 days, after propagating unrooted vegetative cuttings. Plants were propagated and grown entirely in Short Day conditions (13 hours dark).

Propagation:

Type.—Vegetative propagation via stem cuttings.

Time to rooting.—12-14 days with soil temperatures of 18° C.

Rooting habit.—Fine and fibrous.

### Foliage

Compound or single: Compound

Arrangement of Leaves: Alternate

Shape of leaf.—Oblong

Size of leaf.—Width (cm): Near 8 cm Length (cm): Near 9 cm.

Leaf apex.—Mucronate.

Base.—Rounded—cordate.

Attachment.—Petioled.

Aspect.—Slightly undulating.

Margin.—Palmately lobed.

Surface characteristics.—Top: Slightly pubescent Bottom: Pubescent

Petiole:

Colour.—Near 147 A Length: 1.0 to 5 cm

Venation: Net, prominent midvein top and underside.

Colour.—upper side: Near 147 B underside: Near 139 C

Colour: Mature Leaf, upper side: Near 137 A; under side: Near 138 B Young Leaf, upper side: Near 147 A; under side: Near 137 A

### Flower

Flower Appearance: Domed and circular

Flower type: Decorative spider

Flower Form: Incurving

Flower shape: Circular

Flower Habit: Angular

Number of blossoms per branch: Non pinched

Inflorescence form: Cyme

Depth of fully expanded blossoms.—Approx. 5 cm +.

Diameter of fully expanded blossoms.—Approx. 13 to 16 cm.

Sepals:

Number.—17 to 20.

Colour.—Near 137 C.

Length.—0.5 cm.

Width.—Near 0.02 cm.

Texture/Appearance.—Pubescent.

Peduncles: Peduncle length is shortest at the terminal flower and progressively longer moving down the stem; longest length is on average 6 cm; shortest length on average 3 cm.

Length.—4 cm.

Colour.—Near 137 D.

Surface.—Pubescent.

Habit.—Slightly undulating.

Strength.—Medium/strong.

Pedicel:

Length.—Terminal: Near 4 cm; Lateral: Near 6 cm.

Colour.—Near 137 D.

Surface.—Pubescent.

Habit.—Slightly undulating.

Strength.—Medium/strong.

Ray Florets:

Form/Shape.—Incurving.

Texture/Appearance.—Matte.

Number per flower.—200+.

Length.—2.0 to 2.5 cm; Width: 0.4 to 0.8 cm.

Apex.—Spatulate.

Base.—Tapered.

Margin.—Entire.

Disc florets:

Form/shape.—Tubular

Texture/appearance.—Shiny/smooth.

Number per flower.—Approx. 20.

Length.—1 to 5 cm; Width: 0 to 0.5 cm.

Diameter of disc.—1.5 to 2.0 cm.

Fragrance: None

Flower Bud (at onset of colour):

Length.—Near 0.7 cm.

Diameter.—Approx. 1.3 cm.

Form/shape.—Spherical.

General flower colour: 155 B

1. Ray florets, upper side.—Immature: Near 155 A Mature: Near 155 B Older/Fading: Near 155 D.

2. Ray florets, under side.—Immature: Near 155 A Mature: Near 155 B Older/Fading: Near 155 D.

3. Disc florets.—Immature: Near 5 A Mature: Near 5 B Older/Fading. Near 5 D.

4. Bud.—Near 138 C.

Flower progression: Flowers reflex and slightly fade during ageing.

Other Distinguishing Characteristics: None

### Reproductive Organs

Ray Florets: Pistillate

Pistil number.—80+.

Stigma colour.—Near 6 C.

Stigma shape.—Forked

Style colour.—Near 6 C.

Style length.—Near 0.75 cm.

Disc Florets: Pistillate

Pistil number.—Approx. 20.

Stigma colour.—Near 6 C.

Stigma shape.—Forked.

Style colour.—Near 6 C.

Style length.—Near 0.2 cm.

Staminate.—No Observations.

What is claimed is:

1. A new and distinct variety of Chrysanthemum plant, substantially as described and illustrated herein.
